(19) United States
(12) Patent Application Publication
(10) Pub. No.: US 2005/0031717 A1
(43) Pub. Date: Feb. 10, 2005

(54) Salt taste modification

(76) Inventors: John A. DeSimone, Richmond, VA (US); Gerard Laurence Heck, Richmond, VA (US); Vijay Lyall, Midlothian, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/890,398

(22) Filed: Jul. 9, 2004

Related U.S. Application Data
(60) Provisional application No. 60/485,493, filed on Jul. 9, 2003.
(60) Provisional application No. 60/502,622, filed on Sep. 15, 2003.
(60) Provisional application No. 60/525,835, filed on Dec. 1, 2003.

(51) Int. Cl. A61K 35/78; A61K 31/44; A61K 31/21

(52) U.S. Cl. 424/760; 514/358; 514/513; 514/514; 514/680

(57) ABSTRACT

The pharmacology of the capsaicin receptor has been discovered to be predictive of the enhancement of the non-specific salt taste channel. Salt taste in a mammal may be modified by introducing to a mammalian taste receptor cell, a non-salty ligand which is a taste modulator. Examples of the ligand include capsaicin; resiniferatoxin (RTX); piperine; 2-(3,4-dimethylbenzyl)-3-{[(4-hydroxy-3-methoxybenzyl)amino]carbothioyl}propyl pivalate (agonist 23); olvanil, capsiate; evodiamine; ethanol; cetylpyridinium chloride; dodecylpyridinium bromide; capsazepin; SB366791, etc. Salt taste thus may be modified by a non-salty ligand. By introducing certain non-salty ligands into the salt transduction process, the cation non-specific salt taste transduction process may be modified.

Priority is claimed based on the following U.S. provisional application No. 60/485,493 filed Jul. 9, 2003, 60/502,622 filed Sep. 15, 2003, and 60/525,835 filed Dec. 1, 2003, each titled “Class of salt taste modulators.”

### STATEMENT REGARDING FUNDING

Pertinent support may have been received by the National Institute of Deafness and other Communications Disorders Grants DC-02422 (JAD), DC-00122 (JAD) and/or DC-005981-01A2 (VL).

### DESCRIPTION

### FIELD OF THE INVENTION

The present invention is directed to taste, more particularly, to salt taste in mammals.

### BACKGROUND OF THE INVENTION

Saltiness taste conventionally is achieved by the presence of sodium chloride (NaCl), commonly called “salt”. Over time, it was sometimes wanted to provide a salty taste while minimizing the amount of NaCl used, and certain salt substitutes and salt enhancers have been disclosed.

For example, U.S. Pat. No. 4,243,691 issued Jan. 6, 1981, to Mohlenkamp, Jr., for “Sodium-free salt substitute,” disclosed using physiologically-acceptable non-sodium salts for a salty flavor.

U.S. Pat. No. 4,451,494 issued May 29, 1984 to Roan, III, for “Sodium-free salt substitute” taught a mixture of treated animal protein and potassium chloride.

U.S. Pat. No. 4,473,595 issued Sep. 25, 1984 to Rood et al., for “Low-sodium salt substitute.” Sodium chloride was mixed with potassium chloride and magnesium salt.

U.S. Pat. No. 4,560,574 issued Dec. 24, 1985 to Meyer for “Salt substitute containing potassium chloride, maltodextrin and sodium chloride and method of preparation.” See also U.S. Pat. No. 4,556,578, U.S. Pat. No. 4,556,577, and U.S. Pat. No. 4,556,568, all three issued Dec. 3, 1985 to Meyer.

U.S. Pat. No. 4,963,387 (issued Oct. 16, 1990 to Nakagawa et al. for “Salt substitute and foodstuffs containing same”) discloses a salt substitute comprising whey mineral, and which is made from whey and an alkali metal salt (and/or alkaline earth metal salt).

U.S. Pat. No. 5,094,862 (issued Mar. 10, 1992, to Bunick et al. for “Salt substitute granule and method of making same”) discloses a core composition comprising a nonsweet carbohydrate and a coating on the core comprising sodium chloride.

U.S. Pat. No. 5,213,838 (issued May 25, 1993, to Sheikh for “Sodium-free salt substitute containing citrates and method for producing the same”), discloses a sodium-free composition primarily containing potassium citrates and calcium citrates.

U.S. Pat. No. 5,260,091 (issued Nov. 9, 1993 to Locke et al. for “Salt Taste Enhancers”), discloses compositions to use as salt substitutes and enhancers, wherein sodium chloride was mixed with derivatives of amiloride, indanyloxyacetic acid, or anthranilic acid.

Recently, U.S. Pat. No. 6,743,461 (issued Jun. 1, 2004 to Vasquez for “Salt substitute compositions”) disclosed a substitute for table salt, in which is used calcium chloride, a potassium salt, citric acid, rice flour, ginger oil and flavoring.

Such conventional approaches (as mentioned above) for avoiding NaCl use while imparting salt-taste have largely, if not entirely, focused on modifying specific salt taste channels. To the extent that anyone previously has been concerned with the physiological mechanism of salt taste, the main focus has been on the sodium specific ion channel (ENaC, which is blocked by the drug amiloride).

There remains a demand for better methods and products for imparting saltiness as desired by a mammal consuming food or beverage, while avoiding not just NaCl but also being able to avoid use of other salts, such as potassium salts, etc. However, developing new salt-substitutes, salt-enhancers and products relating to salt taste has not been easy, and new products have tended to re-use products already known to have a saltiness effect. There remains a need to identify compounds that have an effect on salt taste but which have not previously been disclosed as being pertinent to salt taste. However, there are shortcomings in the available tools, assays and methodologies for the development of new salt-substitutes, salt-enhancers, and products relating to salt taste. For example, minimizing the need for actual experimentation before a compound can be identified as likely to be pertinent to salt-taste would be advantageous.

### SUMMARY OF THE INVENTION

The present invention exploits the discovery by the inventors that a pharmacological property of a ligand vis-a-vis a capsaicin receptor has a relationship to whether the ligand affects salt taste (namely, by affecting a non-specific salt taste channel). Such a relationship can be exploited, to provide a pre-screening approach for processing ligands which are candidates for salt-taste impact, thereby reducing an amount of actual experimentation needed for locating ligands with usefulness for affecting salt-taste. The present invention also exploits the discovered relationship to manipulate salt-taste via a cation non-specific taste channel, without needing to manipulate salt-taste via a sodium-specific taste channel.

In a preferred embodiment, the invention provides a method of modulating salt taste in a mammal, comprising: introducing to a mammalian taste receptor cell, a non-salty ligand. Examples of such a non-salty ligand are, e.g., an agonist of a vanilloid receptor-1 (VR1) variant taste receptor (such as a pungent agonist (such as capsaicin; resiniferatoxin (RTX); piperine; etc.)); 2-(3,4-dimethylbenzyl)-3-{[(4-hydroxy-3-methoxybenzyl)amino]carbothioyl}propyl pivalate (agonist 23); a non-pungent agonist (such as olvanil, capsiate, evodiamine, etc.); a non-vanilloid VR1 agonist; ethanol; a non-vanilloid, non-VR1 agonist; cetylpyridinium chloride (CPC); dodecylpyridinium bromide; a VR1 antagonist (such as capsazepine; SB366791 (i.e., N-(3-methoxyphenol)-4-chlorocinnamide); etc.); a VR1 agonist (such as a compound including a vanilloid moiety; a compound with a vanilloid moiety replaced with a thiourea moiety; unsaturated dialdehydes; zingerones; indolequinazole alkaloids; allyl isothiocyanates; etc.). Such inventive methods may include: pre-screening at least one non-salty ligand to identify a taste modulator; including isolating RNA necessary for producing VR1 and/or VR1-like channel and incorporating the isolated RNA in an assay system; and/or including an assay system which is a mammalian cell but not a taste receptor; etc. The taste modulator may be an activator of VR1 in a non-taste system. With such inventive methods, optionally salt taste may be modified, such as by a non-pungent VR1 ligand, etc.

In another preferred embodiment, the invention provides a method of modifying a salt transduction process in a mammal, comprising: introducing a non-salty ligand into the salt transduction process, whereby the cation non-specific salt taste transduction process is modified. Optionally in such a salt transduction modification method, whether the cation non-specific salt taste transduction process is modified may be measured with respect to a zero reference point established by a response to Na⁺ cation in the presence of both benzamil and capsazepine, and a ratio of response to Na⁺+benzamil+VR1 modulator to response to Na⁺+benzamil provides a fractional response indicating whether modification has occurred. Examples of an amount in which to introduce the non-salty ligand in such inventive methods is a nanomolar concentration, a micromolar concentration, etc. Optionally, in such inventive methods, along with the non-salty ligand, a cofactor (such as ATP) may be introduced.

Another preferred embodiment of the present invention provides a method of reducing a mammalian subject's salt consumption, comprising: biochemically reducing the subject's taste demand for presence of salt in food, such as, e.g., a method wherein reducing the subject's taste for presence of salt in food is by introducing a non-salty ligand into a salt transduction process in the subject, wherein after non-salty ligand introduction, an amount of salt wanted by the subject in his/her food for taste satisfaction is reduced; a method wherein the subject consumes a non-salty ligand; a method in which a tongue spray with a taste modifier is used; a method in which the subject is a human; etc.

The present invention also includes a preferred embodiment to a method for identifying potential salt taste modifiers, the method comprising: (a) contacting a VR1 ion channel (such as, e.g., a VR1 ion channel that is human) having wild type constitutive activity with at least one test compound; and (b) determining any change from basal activity of the VR1 ion channel having wild type constitutive activity, wherein a change from basal activity of the VR1 ion channel identifies a compound with salt taste modifying activity.

In another preferred embodiment, the invention provides a method for enhancing salt taste, the method comprising: the addition of a potentiating amount of an agonist of VR1 ion channel to a beverage, a foodstuff, or an oral care product e.g., a method in which the bioavailability of the potentiating amount is between 1/100th and 2.5 times the measured EC50 of the agonist in an in-vivo test system or between 1/1000th and 0.25 times the measured EC50 in an in-vitro test system; a method in which the bioavability of such inhibiting amount exceeds 2.5 times the measured EC50 of the agonist in an in-vivo test system or exceeds 0.25 times the measured EC50 in an in-vitro test system; etc.

In an additional preferred embodiment, the invention provides a method for suppressing salt taste, the method comprising: addition of an inhibiting amount of an antagonist of VR1 ion channel to a beverage, a foodstuff, or an oral care product.

Also, another preferred embodiment of the present invention provides a method of predicting whether a candidate ligand will enhance salt taste, comprising: for a candidate ligand, (A) determining pharmacology of the candidate ligand with respect to a capsaicin receptor (such as, e.g., a pharmacology determining step that includes determining whether literature exists specifying the candidate ligand as an agonist or an antagonist for VR1 or TRPV1, such as, e.g., a determination that consists of searching for a literature mention of the candidate ligand being an agonist for VR1 or TRPV 1); and (B) based on the pharmacology of the candidate ligand with respect to the capsaicin receptor, classifying the candidate ligand as likely or not likely to enhance a non-specific salt taste channel. In such inventive methods, location of a literature mention of the candidate ligand being an agonist for VR1 or TRPV1 may be used as a signal of high likelihood that the candidate ligand will enhance a non-specific salt taste channel. Such inventive methods may include finding no literature mention of the candidate ligand being an agonist or an antagonist for VR1 or TRPV1, followed by a step of experimentally testing the candidate ligand (such as, e.g., experimental testing that includes an in-vivo test on cultured cells and/or transfecting a molecular biological host with TRPV1 RNA; experimental testing wherein upon testing the candidate ligand on cultured mammalian dorsal root ganglion (DRG) cells, transient spiking is taken as an indicator of high probability that the candidate ligand will enhance a non-specific salt taste channel; experimental testing wherein upon transfecting a molecular biological host with TRPV1 RNA, at least one of: whether the candidate ligand elicits increases in cellular calcium is determined, and eliciting increase in cellular calcium is taken as an indicator of high probability that the candidate ligand will enhance a non-specific salt taste channel; experimental testing wherein an indicator is selected from: an increase in intracellular sodium and/or a change in membrane potential; etc.)

### BRIEF SUMMARY OF THE DRAWINGS

FIG. 1. Effect of VR1 agonists (RTX and CAP) and CPC on the rat chorda tympani response to NaCl. The tongue was stimulated with a rinse solution (R; 10 mM KCl) and with 100 mM NaCl+10 mM KCl (N) or with 100 mM NaCl+10 mM KCl+5 μM Bz (N+Bz). (A) The Bz-insensitive NaCl chorda tympani response was enhanced (d-e) by 1 μM RTX and inhibited (j-k) by 10 μM RTX. (B) Increasing concentrations of RTX (●), CAP (▪), and CPC (▴) produced biphasic changes in the Bz-insensitive NaCl chorda tympani response. Each point represents the mean±SEM of the normalized chorda tympani response from 3 animals.

FIG. 2. Effect of VR1 antagonists (CZP and SB-366791) on the rat chorda tympani response to NaCl. (A) Chorda tympani responses were recorded during superfusion of the tongue with a rinse solution (10 mM KCl) and a stimulating solution (100 mM NaCl+5 μM Bz+10 μM CZP) containing RTX (0-10 μM). (B) Chorda tympani responses were recorded during superfusion of the tongue with a rinse solution (10 mM KCl) and stimulating solutions containing 10 mM KCl+100 mM NaCl+5 μM Bz+0.75 μM RTX+CZP (0-500 μM). (C) Chorda tympani responses over a range of stimulus temperatures were recorded during superfusion of the tongue with a rinse solution (10 mM KCl) and a stimulating solution (100 mM NaCl+5 μMBz) containing 0 (●), 0.1 μM (▪) and 1 μM (▴) SB-366791. Each point represents the mean±SEM of the normalized chorda tympani response from 3 animals.

FIG. 3. Rat fungiform taste receptor cells contain a VR1 variant transducer. A cDNA library from rat fungiform taste receptor cells was screened for VR1 and its homologs (Liu & Simon, 2001) and yielded a single band of expected size (Lane 1; →). An identical PCR fragment was amplified from rat dorsal root ganglia cDNA (Lane 2). Lane 3=DNA ladder.

FIG. 4. Chorda tympani responses in (A) wildtype mice (WT) and (B) VR1 knockout mice (KO). Tongues were stimulated with 100 mM NaCl (N₂₃°; N₄₂°, subscripts refer to 23° and 42° temperatures, respectively), 100 mM NaCl+5 μM Bz (N+Bz₂₃°; N+Bz₄₂°), and 100 mM NaCl+5 μM Bz+0.75 μM RTX (N+Bz+RTX₂₃°; N+Bz+RTX₄₂°) at either 23° or 42°. Data from 3 wildtype mice and 3 VR1 knockout mice are summarized in (C). Each bar represents the mean±SEM of the normalized chorda tympani response from 3 animals.

FIG. 5. Cation-selectivity and voltage-sensitivity of the amiloride-insensitive channel. (A) CPC induced biphasic changes in rat chorda tympani responses to 100 mM NaCl+5 μM Bz (●), 100 mM NH₄Cl (▴), and 100 mM KCl (▪). The CPC-sensitive chorda tympani responses to KCl and NH₄Cl were obtained by subtracting the maximum suppression value at 10 mM CPC. Each point represents the mean±SEM of the normalized chorda tympani response from 3 animals. (B) Rat chorda tympani responses to 100 mM KCl at zero current-clamp (0 cc), −60 mV and +60 mV voltage-clamp in the absence (left trace) and presence (right trace) of 0.25 mM CPC. (C) Rat chorda tympani responses to 500 mM KCl between −80 and +80 mV lingual voltage-clamp in the absence (▪) and presence of 0.25 mM CPC (●). Each point represents the mean±SEM of the normalized chorda tympani response from 3 animals. (D) Relative changes in [Na⁺]ᵢ in polarized rat fungiform taste receptor cells loaded with Na-green. The changes in [Na⁺]ᵢ are expressed as percent change in fluorescence intensity (F₄₉₀) of Na-green. Values are presented as mean±SEM from 6 regions of interest within the taste bud.

FIG. 6. Effect of external pH (pHₒ) on the NaCl chorda tympani response. (A) Effect of pHₒ (2-10), on the rat chorda tympani response to 100 mM NaCl+5 μM Bz+0.5 μM RTX. Each point represents the mean±SEM of the normalized chorda tympani response from 3 animals. (B) Effect of pHₒ4.7 (▴; N=3), 6.0 (●; N=9), 9.7 (▪; N=6), and ATP (O; N=4) on the temperature-induced chorda tympani response to 100 mM NaCl+5 μM Bz+0.25 μM RTX. Each point represents the mean±SEM of the normalized chorda tympani responses from N, number of animals.

### Detailed Description of a Preferred Embodiment of the Invention

The inventors have discovered and recognized that a non-specific salt taste channel in a mammal may be affected by compounds which traditionally have not been considered “salty ligands”. Examples of compounds which traditionally have been considered “salty ligands” are, e.g., sodium chloride, potassium salts, etc.

Examples of non-salty ligands for use in the present invention also include: agonists of the vanilloid receptor-1 (VR1) variant taste receptor, including pungent agonists of VR1 (such as capsaicin, resiniferatoxin (RTX), piperine, etc.), less-pungent agonists of VR1 (such as 2-(3,4-dimethylbenzyl)-3-{[(4-hydroxy-3-methoxybenzyl)amino]carbothioyl}propyl pivalate (agonist 23), etc.), non-pungent agonists of VR1 (such as olvanil, capsiate, evodiamine, etc.); non-vanilloid VR1 agonists (such as ethanol, etc.); non-vanilloid, non-VR1 agonists (such as cetylpyridinium chloride (CPC), dodecylpyridinium bromide, etc.); VR1 agonists (such as capsazepine, SB366791, etc.); agonists of the classic thermal pain receptor VR1, such as, for example: dihydrocapsaicin; resiniferatoxin; piperine; gingerols; shogaols; olvanil;
N-(4-Hydroxy-3-methoxybenzyl)-N-(2-(4-chlorophenyl)-ethyl)thiourea;
N-(4-Hydroxy-3-methoxybenzyl)-N-(4-chlorobenzyl)thiourea;
N-(4-Hydroxy-3-methoxybenzyl)-N-(2-(4-fluorophenyl)ethyl)thiourea;
N-(E)-(2-(4-Chlorophenyl)ethenyl)-N-(4-hydroxy-3-methoxybenzyl)thiourea;
N-(Z)-(2-(4-Chlorophenyl)ethenyl)-N-(4-hydroxy-3-methoxybenzyl)thiourea;
N-(2-(2,4-Dichlorophenyl)ethyl)-N-(4-hydroxy-3-methoxybenzyl)thiourea;
N-(4-Hydroxy-3-methoxybenzyl)-N-(2-phenylethyl)thiourea;
N-(3-(4-Chlorophenyl)propyl)-N-(4-hydroxy-3-methoxybenzyl)thiourea;
N-(4-tert-Butylbenzyl)-N-(4-hydroxy-3-methoxybenzyl)-thiourea (Ii); N-(4-(2-Aminoethoxy)-3-methoxybenzyl)-N-(2-(4-chlorophenyl)ethyl)thiourea;
N-(4-(2-Aminoethoxy)-3-methoxybenzyl)-N-(2-phenylethyl)thiourea;
N-(4-(2-Phthalimidoethoxy)-3-methoxybenzyl)-N-(2-(4-fluorophenyl)ethyl)thiourea;
N-(4-(2-Aminoethoxy)-3-methoxybenzyl)-N-(2-(4-fluorophenyl)ethyl)thiourea;
N-(4-(2-Aminoethoxy)-3-methoxybenzyl)-N-(2-(2,4-dichlorophenyl)ethyl)thiourea;
N-(4-(2-Aminoethoxy)-3-methoxybenzyl)-N-(4-iodobenzyl)thiourea;
N-(4-(2-Aminoethoxy)-3-methoxybenzyl)-N-(4-(trimethylsilyl)benzyl)thiourea;
N-(4-(2-Aminoethoxy)-3-methoxybenzyl)-N-(4-tert-butylphenyl)thiourea;
N-(4-(2-Aminoethoxy)-3-methoxybenzyl)-N-(4-tert-butylbenzyl)thiourea;
N-(4-(2-Aminoethoxy)-3-methoxybenzyl)-N-(3,5-di-tertbutylbenzyl)thiourea;
N-(4-(2-Aminoethoxy)-3-methoxybenzyl)-N-(2-(4-tertbutylphenyl)ethyl)thiourea;
N-(4-(3-Aminopropoxy)-3-methoxybenzyl)-N-(2-(4-chlorophenyl)ethyl)thiourea;
N-(3-Methoxy-4-(2-(methylamino)ethoxy)benzyl-N-(2-(4-chlorophenyl)ethyl)thiourea;
N-(4-(2-(N,N-Dimethylamino)ethoxy)-3-methoxybenzyl)-N-(2-(4-chlorophenyl)ethyl)thiourea;
N-(4-(2-(Trimethylammonio)ethoxy)-3-methoxybenzyl)-N-(2-(4-chlorophenyl)ethyl)thiourea trifluoroacetate;
N-(4-(2-Phthalimidoethoxy)-3-methoxybenzyl)-N-(2-(4-chlorophenyl)ethyl)thiourea;
N-(4-(2-Acetamidoethoxy)-3-methoxybenzyl)-N-(2-(4-chlorophenyl)ethyl)thiourea;
N-(4-(2-((Ethoxycarbonyl)amino)ethoxy)-3-methoxybenzyl)-N-(2-(4-chlorophenyl)ethyl)-thiourea;
N-(4-(2-(Boc-amino)ethoxy)-3-methoxybenzyl)-N-(2-(4-chlorophenyl)ethyl)thiourea;
9,13,14-Orthophenylacetylresiniferonyl 20-(3-Azido-4-methoxyphenylacetate);
N-(9,13,14-Orthophenylacetylresiniferonyl)-4-hydroxy-3-methoxyphenylacetamide 11;
9,13,14-Orthophenylacetylresiniferonyl 20-[4-(Aminoethoxy)-3-methoxyphenylacetate];
9,13,14-Orthophenylacetylresiniferonyl 20-Phenylacetate;
9,13,14-Orthophenylacetylresiniferonyl 20-(3-Methoxyphenylacetate);
9,13,14-Orthophenylacetylresiniferonyl 20-(3,4-Dimethoxyphenylacetate);
9,13,14-Orthophenylacetylresiniferonyl 20-Acetate;
9,13,14-Orthophenylacetylresiniferonyl 20-Nonanoate;
9,13,14-Orthoacetylresiniferonyl 20-(4-Hydroxy-3-methoxyphenylacetate);
9,13,14-Orthobenzoylresiniferonyl 20-(4-Hydroxy-3-methoxyphenylacetate);
9,13,14-Orthophenylacetyl-3,-hydroxyresiniferonyl 20-(4-Hydroxy-3-methoxyphenylacetate);
9,13,14-Orthophenylacetyl-4,-methoxyresiniferonyl 20-(4-Hydroxy-3-methoxyphenylacetate);
12-Deoxyphorbol 13-Phenylacetate 20-(4-Hydroxy-3-methoxyphenylacetate);
Phorbol 12,13-Diacetate 20-(4-Hydroxy-3-methoxyphenylacetate);
Phorbol 12,13-Didecanoate 20-(4-Hydroxy-3-methoxyphenylacetate);
   sesquiterpenoid unsaturated dialdehydes; warburganal; polygodial; isovelleral; merulidial; isotadeonal; ginesosides; nonivamide [nonanamide, N-(4-hydroxy-3-methoxyphenyl)methyl]; nonpungent capsiate non-agonists; nordihydrocapsiate 4-hydroxy-3methoxybenzyl 7-methyloctanoate; phorboid 20-homovanillates; Phorbol 12-Phenylacetate 13-Acetate 20-Homovanillate (PPAHV); Phorbol 12-benzoate 13-acetate 20-homovanillate; Phorbol 12-p-azidophenylacetate 13-acetate 20-homovanillate; Phorbol 12-p-azidobenzoate 13-acetate 20-homovanillate; Phorbol 12-cyclohexylacetate 13-acetate 20-homovanillate; Phorbol 12-cyclohexanecarboxylate 13-acetate 20-homovanillate; Phorbol 12,13-Bis(phenylacetate) 20-Homovanillate; 12-Dehydrophorbol 13-Acetate 20-Homovanillate; 12-Dehydrophorbol 13-Phenylacetate 20-Homovanillate; (3R)-Dihydrophorbol 12-Phenylacetate 13-Acetate 20-Homovanillate; Phorbol 12-Phenylacetate 13-Acetate 20-Acetylhomovanillate; N-acylethanolamines; Anandamide (arachidonoylethanolamide); palmitoylethanolamide (PEA); glycerol nonivamide; stearoyl vanillylamide; unsaturated long-chain N-Acyl-vanillyl-amides (N-AVAMS); zingerone; compounds which are analogues or derivatives of above-mentioned agonists and/or compounds (such as capsaicin-like agonists, capsaicin analogues, etc.); etc. The above-named compounds have previously been known (e.g., as agonists of the classic thermal pain receptor VR1, etc.) and may be obtained or synthesized as is known in the art. Compounds not yet in existence or presently undiscovered may be within the non-salty ligands of this invention.

Preferably, the non-salty ligands to use in inventive salt-taste applications (such as a salt enhancer, salt substitute, etc.) are less-pungent or non-pungent VR1 ligands, with a VR1 ligand that is a pungent stimulus for trigeminal nerve sensation being avoided, i.e., the capsaicin burn from chili peppers is usually undesirable.

An amount for including the non-salty ligands of the invention in foodstuffs or consumer products is an amount sufficient to elevate salt taste sensitivity, or to allow for reduction in added or processing sodium, or to block salt taste, with examples of an amount being, e.g., a micromolar concentration, a nanomolar concentration, etc.

Additionally, by recognizing that compounds which traditionally have not been considered “salty ligands” may be able to affect salt-taste in mammals by affecting a non-specific salt taste channel, the present invention introduces new methods, systems, and products for salt substitutes, salt enhancers and the like for food and beverages, such as, e.g., by providing new salt-taste-related uses for existing compounds. It will be appreciated, however, that the present invention is not limited to known compounds, and that screening methods and other methods according to the present invention may be applied to analyze and/or classify compounds that are prospectively disclosed.

The present invention includes an embodiment in which a pre-screening approach is provided for processing ligands which are candidates for salt-taste impact, thereby reducing an amount of actual experimentation needed for locating ligands with usefulness for affecting salt-taste.

Another use of the present invention is to manipulate salt-taste via a non-specific taste channel, without needing to manipulate salt-taste via a specific taste channel.

The following inventive Examples are mentioned, but it will be appreciated that the invention is not limited to the Examples.

### EXAMPLE 1

Capsaicin has been shown to modulate salt taste biphasically, increasing responses to sodium, potassium, and ammonium salts at low levels of the capsaicin ligand and blocking responses to these salts at higher concentrations. Measurements were made of the chorda tympani response in rat to 100 mM NaCl+5 μM benzamil, 100 mM KCl, and 100 mM NH₄Cl at capsaicin concentrations varying from 1 μM to 1 mM. The response to NaCl+benzamil increased with increasing capsaicin concentration up to 40 μM. At higher capsaicin concentrations the response decreased and by 200 μM capsaicin, the response to NaCl+benzamil was eliminated. KCl and NH₄Cl behaved similarly except that for these salts a residual response remained at 200 μM capsaicin.

### EXAMPLE 2

The antagonist of VR1, capsazepine, has been shown to strictly block vanilloid-enhanced taste responses from sodium, potassium, and ammonium salts without demonstrating a response-increasing concentration range. Concentrations of capsazepine ranging from 0-500 μM, monotonically reduced the magnitude of the chorda tympani response to 100 mM NaCl+5 μM benzamil+0.75 μM RTX ultimately to zero.

### EXAMPLE 3

A rule has been developed that will predict ligands which modulate salt taste. The rule is as follows. Agonists or antagonists of the VR1 receptor will act respectively as enhancers and suppressors of the salt response of taste receptor cells. This rule can be operationalized by isolating the RNA necessary for producing the VR1 and VR1-like channel and incorporating it in an assay system such as frog oocytes or human embryonic kidney cells.

### EXAMPLE 4

The amiloride-insensitive salt taste receptor is the predominant transducer of salt taste in some mammalian species, including humans. In this Example 4, the physiological, pharmacological and biochemical properties of the amiloride-insensitive salt taste receptor were investigated by RT-PCR, by the measurement of unilateral apical Na⁺ fluxes in polarized rat fungiform taste receptor cells and by chorda tympani taste nerve recordings. The chorda tympani responses to NaCl, KCl, NH₄Cl, and CaCl₂ were recorded in Sprague Dawley rats, and in wildtype and vanilloid receptor-1 (VR1) knockout mice. The chorda tympani responses to mineral salts were monitored in the presence of vanilloids (resiniferatoxin and capsaicin), VR1 antagonists (capsazepine and SB-366791), and at elevated temperatures.

Introduction

Mammals utilize two types of taste receptors to detect mineral salts: one that is Na⁺ specific (said to be amiloride-sensitive because it is inhibited pharmacologically by the drug, amiloride), and a second that does not discriminate among Na⁺, K⁺, and NH₄⁺ (said to be amiloride-insensitive). In humans, salty taste perception is predominantly amiloride-insensitive so a better understanding of this taste receptor is desired.

Previously we identified an apical amiloride-insensitive cation pathway in rat fungiform taste receptor cells that is modulated by cetylpyridinium chloride (CPC). DeSimone J A, Lyall V, Heck G L, Phan T H T, Alam R I, Feldman G M et al. (2001), A novel pharmacological probe links the amiloride-insensitive NaCl, KCl, and NH₄Cl chorda tympani taste responses, J Neurophysiol 86, 2638-2641. Using a rat model and a vanilloid receptor-1 (VR1) knockout mouse model (Caterina M J, Leffler A, Malmberg A B, Martin W J, Trafton J, Petersen-Zeitz K R et al. (2000), Impaired nociception and pain sensation in mice lacking the capsaicin receptor, Science 288, 306-313), the CPC-sensitive, amiloride-insensitive taste receptor is demonstrated to be a non-selective cation channel that has functional similarities with cloned VR1 and is derived from the VR1 gene.

Methods

Chorda tympani nerve recordings. Recordings from the chorda tympani (CT) taste nerves of anesthetized rats were made. Recordings were made with various salt stimuli and with the taste receptive field under current or voltage clamp in order to observe the effect, if any, of modulating the driving force for ions through putative were made apical membrane ion channels involved in salt taste reception.

CT responses were also monitored in Wildtype C57BL/6J and homozygous VR1 knockout B6.129S4-Trpv1^{tmljul} mice (Jackson Laboratories).

[Na⁺]ᵢ measurement in polarized taste receptor cells. A small piece of the anterior lingual epithelium containing a single fungiform papilla was mounted in a special microscopy chamber (Lyall et al., 2002, supra). Relative changes in intracellular sodium activity ([Na⁺]ᵢ) were monitored in polarized taste receptor cells with the sodium sensitive fluoroprobe sodium-green. The taste receptor cells in the taste bud were visualized from the basolateral side through a 40× objective using a fluorescence microscope.

RT-PCR. Taste buds were harvested from rat fungiform papillae, aspirated with a micropipette and individually transferred onto coverslips, avoiding contaminating cells and debris (Vinnikova A K, Alam R I, Malik S A, Ereso G L, Feldman G M, McCarty J M et al. (2003), Na+—H+ exchange activity in taste receptor cells, J Neurophysiol (2003), published online (Nov. 5, 2003),10.1152/jn.00809.2003). Total RNA was extracted using a RNeasy Mini kit (Qiagen) with incorporation of the DNAse digestion step. The cDNA was generated and amplified using a Super SMART™ PCR cDNA Synthesis Kit (Clontech) according to the manufacturer's protocol. PCR screening of the fungiform cDNA for the presence of VR1 and its homologs was performed with HotStarTaq Poymerase (Qiagen) using primers and conditions described elsewhere (Liu & Simon, 2001). The PCR products were analyzed by agarose gel electrophoresis. Bands of the predicted size were purified using the MinElute Gel Extraction kit (Qiagen) and directly sequenced.

Results

To investigate specifically the amiloride-insensitive salt taste receptor, rat and mouse CT responses to mineral salts in the presence of benzamil (Bz), a more specific ENaC inhibitor than amiloride, were monitored.

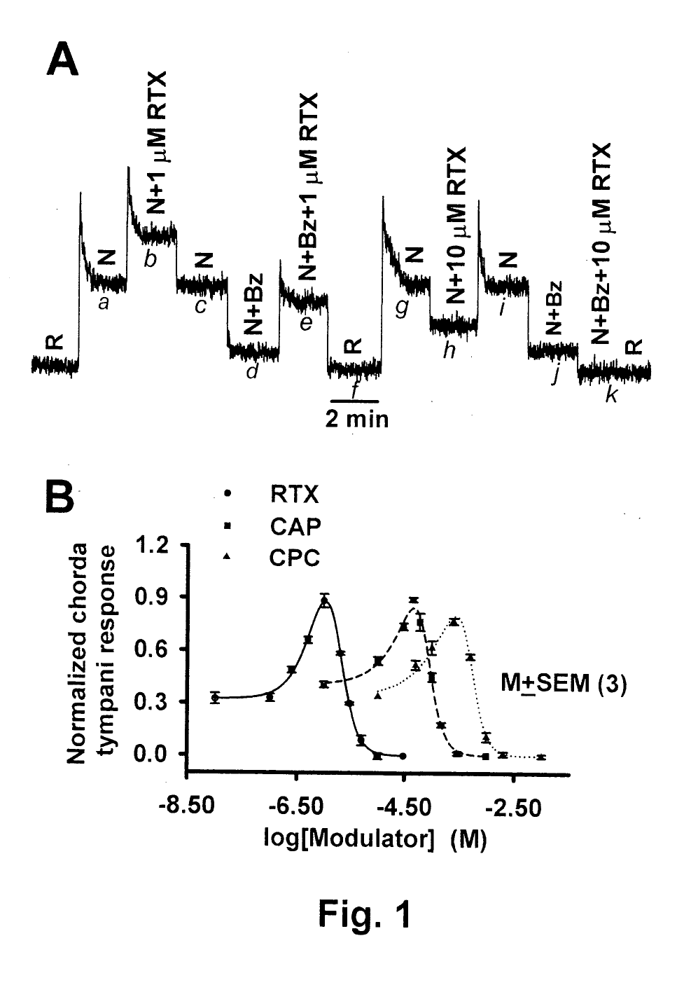

Vanilloids and VR1 antagonists modulate rat CT responses to mineral salts. The effect of VR1 agonists, resiniferatoxin (RTX) and capsaicin (CAP), was investigated on the NaCl chorda tympani responses. Stimulating the tongue with 100 mM NaCl+1 μM RTX reversibly increased the chorda tympani response compared to 100 mM NaCl alone (FIG. 1A; a-b-c). The ENaC component of the chorda tympani response was blocked by 5 μM Bz c-d). Superfusing the tongue with 100 mM NaCl+5 μM Bz+1 μM RTX gave the same magnitude of enhancement observed without Bz (FIG. 1A; d-e vs a-b). In contrast, stimulating with 100 mM NaCl+10 μM RTX reversibly decreased the chorda tympani response (g-h-i) compared to 100 mM NaCl alone (f-g). In the next step, the ENaC component was again blocked with 5 μM Bz (i-j). Stimulating with NaCl+5 μM Bz+10 μM RTX reduced the response (j-k) to rinse level; 10 mM KCl). The data suggest that depending upon its concentration, RTX acts both as an agonist and antagonist of the Bz-insensitive NaCl chorda tympani response.

The rat Bz-insensitive NaCl chorda tympani response over a range of RTX, CAP and CPC concentrations gave bell-shaped concentration-response curves (FIG. 1B). RTX caused the chorda tympani response to NaCl (100 mM NaCl+5 μM Bz) to increase monotonically between 0.1 μM and 1 μM, at higher RTX concentrations the NaCl response decreased reaching control levels around 3 μM. Above 3 μM RTX, the NaCl chorda tympani response was less than control, reaching rinse levels around 10 μM RTX concentration (FIG. 1B). CAP, also a VR1 agonist, was similarly effective, although at relatively higher concentrations. Maximum activation of the NaCl chorda tympani response occurred around 40 μM CAP, and by 200 μM responses decreased to the rinse level (FIG. 1B). CPC, a compound previously shown to modulate the amiloride-insensitive chorda tympani response to mineral salts (DeSimone et al., 2001, supra), demonstrated a similar bimodal effect on the NaCl chorda tympani response. Maximum activation of the NaCl chorda tympani response occurred around 250 μM CPC, and by 2 mM the response decreased to rinse level (FIG. 1B). In the presence of 5 μM Bz, addition of RTX (10 μM), CAP (200 μM), or CPC (2 mM) reduced the NaCl chorda tympani response to a baseline level that was indistinguishable from the rinse level. This suggests that the NaCl chorda tympani response is composed entirely of a Bz-sensitive component (ENaC) and a second Bz insensitive component that is modulated by RTX, CAP, and CPC with a rank order potency of RTX>CAP>CPC.

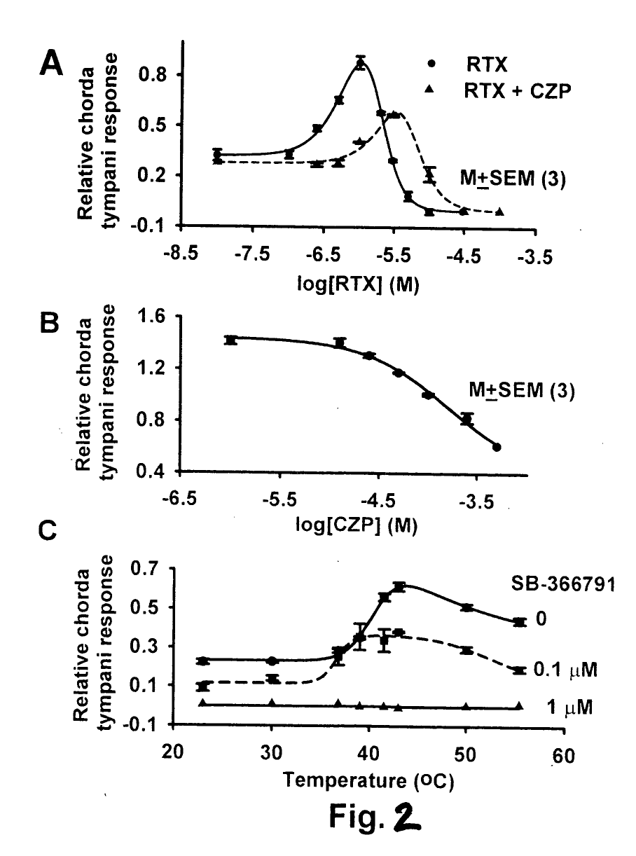

The VR1 antagonists, capsazepine (CZP) and SB-366791, inhibited the effects of vanilloids, CPC, and temperature on Bz-insensitive NaCl chorda tympani responses. CZP (10 μM) decreased the magnitude of the maximum Bz-insensitive NaCl chorda tympani response and shifted the RTX concentration response curve to the right (FIG. 2A). Stimulating the tongue with NaCl solutions containing a fixed RTX concentration but increasing concentrations of CZP demonstrated a dose-dependent inhibition of the NaCl chorda tympani response (FIG. 2B). At a CZP concentration of 100 μM, chorda tympani responses decreased to the level of chorda tympani responses with NaCl alone. At CZP concentrations of 250 μM and above, the chorda tympani response was below the level of the chorda tympani response with NaCl alone. A more specific VR1 antagonist, SB-366791, blocked the temperature induced effects on the chorda tympani response to NaCl+Bz in a dose dependent manner. At 1 μM, SB-366791 completely blocked the Bz-insensitive NaCl chorda tympani response at 23° and at elevated temperatures (FIG. 2C).

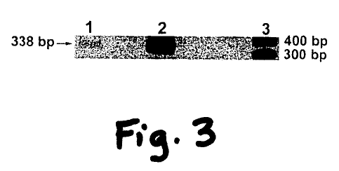

The amiloride-insensitive salt taste receptor is non-functional in VR1 knockout mice. The Bz-insensitive NaCl chorda tympani response is modulated by RTX, CAP and elevated temperature. The VR1 antagonists, CZP and SB-366791, inhibit the effect of vanilloids, CPC, and temperature on the Bz-insensitive NaCl chorda tympani response. Multiple stimuli produced an integrated effect on the Bz-insensitive NaCl chorda tympani response. The above results demonstrate that the amiloride-insensitive salt taste receptor has functional similarities with the VR1 receptor. Consistent with this, a VR1 mRNA transcript common to several channels in the Transient Receptor Potential (TRP) receptor family was detected in rat fungiform taste receptor cells. We constructed a cDNA library from fungiform taste buds. Using primers sense 5′-TGAAAAACACCGTTGGGGAC-3′ SEQ ID#1, and antisense 5′-GTAGACGAACATAAACCGGC-3′ SEQ ID #2 (Liu L & Simon SA (2001), Acidic stimuli activates two distinct pathways in taste receptor cells from rat fungiform papillae, Brain Res 923, 58-70), a single band of 338 bp was obtained (FIG. 3; lane 1), that yielded 100% homology with TRP channels: rVR1, rVRL-1, rSIC, and rVR5′sv. As a positive control, an identical PCR fragment was also amplified from a rat dorsal root ganglion cDNA library, known to contain the VR1 transcript (FIG. 3; lane 2).

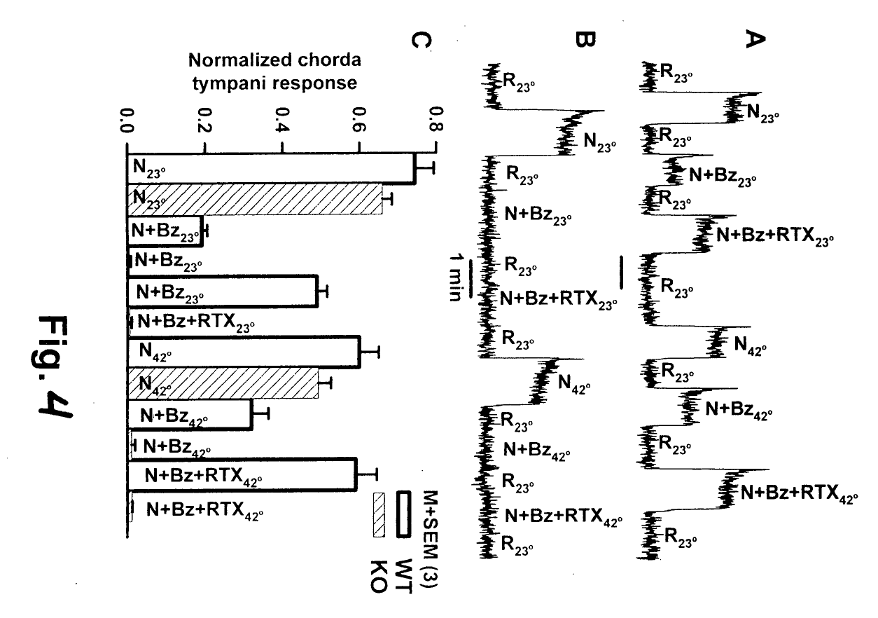

Homozygous VR1 knockout mice, B6.129S4-Trpv1^{tmljul} (Caterina et al., 2000, supra) demonstrated no Bz-insensitive NaCl chorda tympani response and no sensitivity to vanilloids and temperature. At 23°, in wildtype C57BL/6J mice, about 25% of the NaCl chorda tympani response (N23′) was Bz-insensitive (N+Bz₂₃°) (FIG. 4A). Its magnitude was enhanced by RTX (N+Bz+RTX₂₃′). At 42° the Bz-insensitive NaCl chorda tympani response (N+Bz₄₂′) and the response to RTX (N+Bz+RTX₄₂°) were significantly enhanced relative to 23°. The absence of a Bz-insensitive NaCl chorda tympani component indicates that in VR1 null mice the entire NaCl response is composed of a Bz-sensitive ENaC component ((N₄₂°(N+Bz₄₂°); FIG. 4B). No effects of RTX and elevated temperature were observed in response to NaCl+Bz (N+Bz) and to NaCl+Bz+RTX (N+Bz+RTX) in VR1 knockout mice (FIG. 4B). The data from 3 wildtype and 3 VR1 knockout mice are summarized in FIG. 4C. In FIG. 4C, in VR1 knockout mice, the magnitude of the chorda tympani responses with N+Bz₂₃′, N+Bz+RTX23°,N+Bz₄₂° and N+Bz+RTX₄₂° were not significantly different from zero (p>0.05; N=3). The absence of a constitutively active Bz-insensitive NaCl chorda tympani response in VR1 knockout mice demonstrates that the amiloride-insensitive salt taste receptor is derived from the VR1 gene, and is most likely a homolog of VR1.

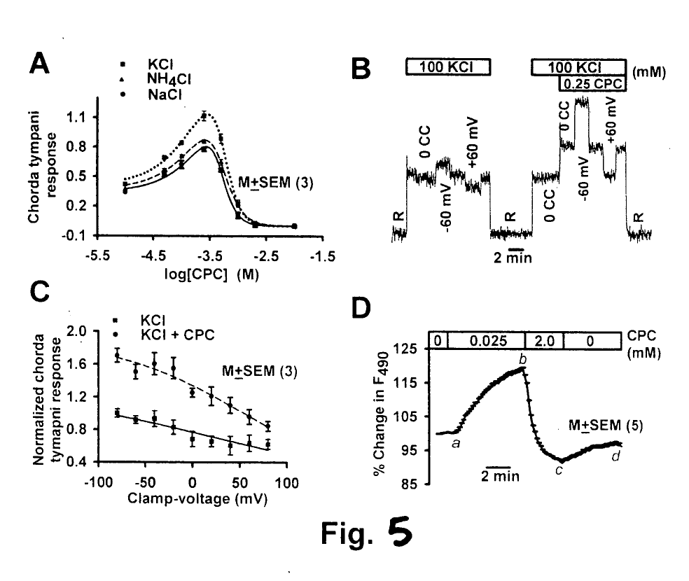

The amiloride-insensitive salt taste receptor is a non-selective cation channel. Stimulating the tongue with mixtures of CPC plus 100 mM KCl or NH₄Cl (FIG. 5A) produced bell shaped concentration-response relationships similar to those obtained with CPC+NaCl+Bz. RTX and CAP also produced similar bell shaped responses with NaCl, KCl, NH₄Cl, and CaCl₂ with the same rank order potency as obtained for NaCl+Bz.

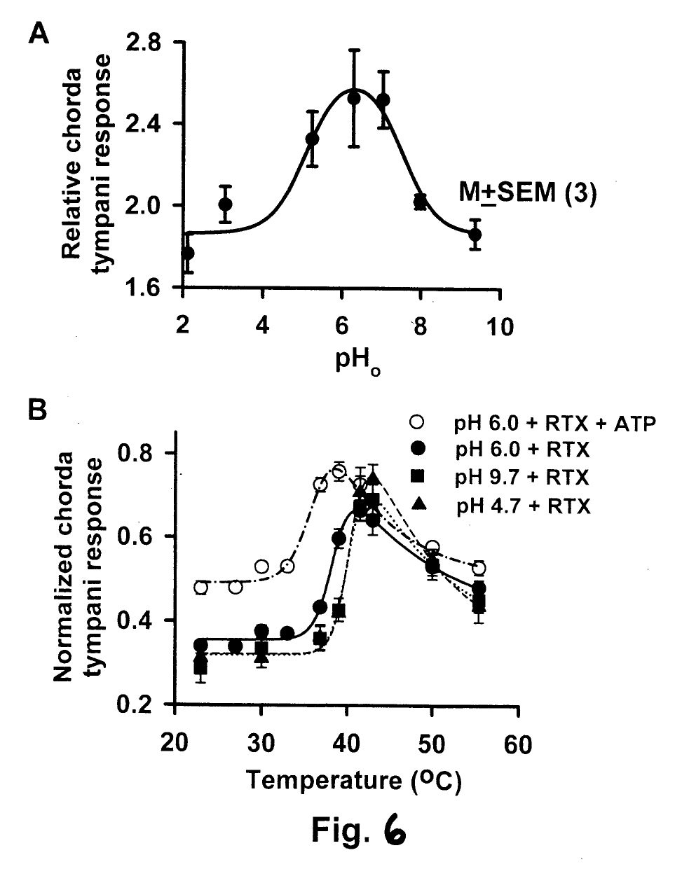

Agonist concentrations of vanilloids and CPC and elevated temperature increased the apical conductance to cations. The rat chorda tympani response to 100 mM KCl was slightly enhanced at −60 mV lingual voltage-clamp (referenced to the oral cavity) and slightly suppressed at +60 mV (FIG. 5B). In the presence of 0.25 mM CPC, the same voltages exerted significantly larger effects on the response (FIG. 6B). FIG. 5C shows the chorda tympani response to KCl under control conditions and after CPC treatment as a function of clamp voltages between −80 mV and +80 mV. In the presence of CPC, the mean value of the normalized chorda tympani response at −80 mV, 0 and +80 mV was significantly greater (p<0.01; paired; N=3; FIG. 5C) than its corresponding value in the absence of CPC. Thus the CT response to KCl across voltages show that both the response and the slope of the response with voltage increased between −80 mV and +80 mV in the presence of CPC. If the amiloride-insensitive salt taste receptor is an apical cation channel, the CT response is expected to be proportional to the cation flux through apical conductances. Therefore, any agent that increases the response must do so by increasing the conductance of the cation channel transducers. This effect is observed in FIG. 5C where, CPC, is seen to increase both response and response conductance (i.e. slope of the response with voltage). The presence of an apical CPC-modulated Na⁺ pathway was confirmed directly by the measurement of the apical Na⁺ flux in polarized fungiform TRCs. At an agonist concentration, CPC enhanced (FIG. 5D; a-b), and at an antagonist concentration, CPC inhibited (FIG. 5D; b-c) the unilateral apical Na⁺ flux.

Physiological and pharmacological properties not shared by the amiloride insensitive salt taste receptor with VR1. Besides some similarities, there are also significant physiological differences between cloned VR1 and the amiloride insensitive salt taste receptor. VR1 is activated by a decrease in external pH (pHₒ). In the absence of a ligand, the Bz-insensitive NaCl chorda tympani response was not affected by changes in pHO. However, the RTX-induced change in the Bz-insensitive NaCl chorda tympani response described a bell shaped curve as a function of stimulus solution pHₒ (FIG. 6A). Similar to its effects on VR1, RTX induced the greatest increase in the Bz-insensitive NaCl chorda tympani response under moderately acidic conditions (pH 6).

Adenosine 5′-triphosphate (ATP) decreases the temperature threshold for VR1 (Tominaga et al., 2001). ATP (500 μM) alone had no effect on the temperature threshold of the Bz-insensitive NaCl chorda tympani response. In the presence of 0.25 μM RTX (FIG. 6B), ATP (500 μM) decreased t_{0.5} from 37.9°±0.2° (N=9) to 35.3°±0.80 (p<0.05; N=4). Thus ATP, while not effective alone, increases the effectiveness of the vanilloid as a salt taste enhancer.

Similarly, changes in pHO between 4.7 and 9.7 had no effect on the temperature threshold of the Bz-insensitive NaCl chorda tympani response in the absence of an agonist. However, in the presence of 0.25 μM RTX, changing pHO from 6.0 to pH 4.7 or to 9.7 (FIG. 6B) increased t_{0.5} from 37.9°±0.2° (N=9) to 40.0°±0.01° (N=3) or to 39.7°±0.02° (N=6; p<0.05), respectively. Thus at a moderately acidic pH of 6.0, both pHₒ and RTX act synergistically to lower the temperature threshold of the Bz-insensitive NaCl chorda tympani response.

Results

These studies involving in vivo chorda tympani recordings and apical ion flux measurements in polarized fungiform taste receptor cells in vitro demonstrate that the amiloride-insensitive salt taste receptor is a non-selective cation channel that is permeable to Na⁺, K⁺, NH₄⁺ and Ca²⁺ ions. The amiloride-insensitive cation channel is a member of the TRP channel family. It demonstrates functional similarities to the VR1 receptor. It is modulated by vanilloids, temperature, and VR1 antagonists and can integrate the effect of multiple stimuli.

However, there are also significant differences between VR1 and the amiloride-insensitive salt taste receptor. In contrast to VR1, the amiloride-insensitive cation channel is constitutively active in the absence of a ligand at 23°, and is not modulated by pHₒ and ATP.

The specificity of the channel as a transducer in salt taste is demonstrated by the observations that RTX has no effect on the CT responses to sucrose, quinine or H⁺ ions. The channel is non-functional in VR1 knockout mice (FIGS. 4B and 4C). VR1 null mice demonstrate no amiloride-insensitive NaCl CT component and no salt taste sensitivity to RTX and temperature (FIGS. 4B and 4C). The VR1-variant salt taste receptor is responsible for mineral salt detection. It accounts for the entire amiloride-insensitive chorda tympani response to NaCl (FIG. 1) and part of the response to K⁺, NH₄⁺ (FIG. 5A) and Ca²⁺ salts.

The amiloride-insensitive cation channel activity increases in parallel with temperature (FIGS. 2C, 4A) and with the additive effects of RTX and RTX+ATP (FIG. 6B). This indicates that the agonists and potentiators of the amiloride-insensitive cation channel interact to reduce the temperature threshold of the channel. This allows for increased salt taste sensitivity without an increase in temperature.

The amiloride-insensitive salt taste receptor may also play an important role in detecting Na⁺ while ingesting foods that are acidic. In mixtures containing NaCl and acidic stimuli, acid equivalents enter taste receptor cells and decrease pHᵢ, inhibiting Na⁺-influx through the amiloride-sensitive ENaC and hence inhibiting the NaCl chorda tympani response (Lyall V, Alam R I, Phan THT, Russell O F, Malik S A, Heck G L et al. (2002), Modulation of rat chorda tympani NaCl responses and intracellular Na⁺ activity in polarized taste receptor cells by pH, J Gen Physiol 120, 793-815). Thus, in acid/salt mixtures, ENaC is inhibited and does not contribute to the overall salt taste.

Unlike the amiloride-sensitive ENaC, the constitutively active amiloride-insensitive cation channel is insensitive to external pH. However, low pHₒ increases its sensitivity to vanilloids (FIG. 6A) and decreases the temperature threshold of the amiloride-insensitive cation channel (FIG. 6B). This suggests that in acid/salt mixtures salt taste is transduced predominantly by the amiloride-insensitive salt taste receptor.

The differential contribution of the amiloride-sensitive ENaC and the amiloride-insensitive cation channel to overall salt taste varies widely across species. In humans (Feldman G M, Mogyorosi A, Heck G L, DeSimone J A, Santos C R, Clary R A et al. (2003), Salt-evoked lingual surface potential in humans, J Neurophysiol 90, 2060-2064; Halpern BP (1998), Amiloride and vertebrate gustatory responses to NaCl, Neurosci Biobehav Rev 23, 5-47), the major mechanism mediating salt taste is amiloride-insensitive. The modulation of the amiloride insensitive salt taste receptor by vanilloid and non-vanilloid compounds, suggests that specific salt taste suppressors and enhancers for humans may be provided. Such compounds may be useful in the management of hypertension and cardiovascular disease.

In summary, Na⁺ transport across fungiform taste receptor cells occurs through both cellular and transcellular pathways. In the apical membranes of taste receptor cells salt taste transduction involves a Na⁺-specific receptor, the apical amiloride-sensitive ENaC and a VR1 variant non-specific cation channel that is amiloride- and Bz-insensitive, resulting in the apical influx of Na⁺ into taste receptor cells (Lyall et al., 2002, supra). The exit of Na⁺ from taste receptor cells occurs via the basolateral Na⁺-K⁺ ATPase. An additional Na⁺ transport mechanism involves the basolateral Na⁺-H⁺ exchanger isoform 1 (NHE-1) (Vinnikova et al., 2003, supra). The apical Na⁺-H⁺ exchanger isoform 3 (NHE-3) seems to be quiescent (Id.). The transcellular transport of Na⁺, K⁺, NH₄⁺, and Ca²⁺ ions occurs via the paracellular shunt mechanism and is anion-dependent.

The results in this Example 4 indicate that the amiloride-insensitive salt taste receptor is a constitutively active non-selective cation channel derived from the VR1 gene. It accounts for all of the amiloride-insensitive CT taste nerve response to Na⁺ salts and part of the response to K⁺, NH₄⁺, and Ca²⁺ salts. It is activated by vanilloids and temperature (>38°), and is inhibited by VR1 antagonists. In the presence of vanilloids, external pH and ATP lower the temperature threshold of the channel. This allows for increased salt taste sensitivity without an increase in temperature. VR1 knockout mice demonstrate no functional amiloride-insensitive salt taste receptor and no salt taste sensitivity to vanilloids and temperature.

### EXAMPLE 5

In this Example 5, the effect of ethanol on the amiloride-insensitive salt taste receptor was investigated by direct measurement intracellular Na⁺ ([Na⁺]ᵢ) by fluorescence imaging in polarized fungiform TRCs and by CT taste nerve recordings. The CT responses to KCl and NaCl were recorded in rats, and in wild-type and VR1 knockout mice, in the presence of VR1 agonists (ethanol, resiniferatoxin and elevated temperature) and VR1 antagonists (casazepine and SB-366791). In the absence of mineral salts ethanol elicited only transient phasic CT responses. In the presence of mineral salts ethanol produced CT responses that are similar to salt responses and increased apical cation flux in TRCs. At a concentration of <50%, ethanol enhanced, and >60% concentration, inhibited CT responses to KCl and NaCl were seen.

Whether ethanol acts as an agonist of the amiloride-insensitive salt taste receptor was tested, including testing if ethanol modulates the interactions of the amiloride-insensitive salt taste receptor with RTX and elevated temperature, classical agonists of the amiloride-insensitive salt taste receptor, and if the ethanol-induced effects on the taste receptor can be inhibited by the VR1 antagonists CZP and SB-366791. CT responses were monitored in two animal models: a rat model and in the VR1 knockout (KO) mouse model (see, e.g., Caterina et al., 2000, supra). The CT responses were monitored while the tongue was stimulated with mineral salts in mixtures with ethanol and specific agonists and antagonists of the VR1 receptor. The results indicate that ethanol acts both as agonist and an antagonist of the VR1 variant cation channel in fungiform TRCs. At concentrations less than 50% ethanol behaves as an agonist for 100 mM NaCl. The VR1 KO mice lack the amiloride- and Bz-insensitive component of the NaCl CT response and were insensitive to ethanol. Ethanol was shown to modulate salt responses by its action on the VR1 variant cation channel in fungiform TRCs.

Results

In Vitro Studies

Effect of ethanol on the unilateral apical Na⁺ flux in polarized fungiform TRCs. The effect was observed of increasing apical Na⁺ concentration on F₄₉₀ of sodium-green loaded TRCs in the absence and presence of Bz and SB-366791. An increase in apical Na⁺ concentration from 0 to 150 mM increased F₄₉₀ reversibly. A change in apical Na⁺ from 0 to 150 mM in the presence of 5 mM Bz produced a significantly smaller increase in F₄₉₀ relative zero Bz. In the next step, an increase in apical Na⁺ concentration in the presence of 5 mM Bz+1 mM SB-366791 completely inhibited the increase in F₄₉₀. These results demonstrate that in fungiform TRCs, apical Na⁺ entry occurs through Bz-sensitive ENaCs and by Bz-insensitive, but SB-366791-sensitive VR1 variant cation channels.

The amiloride- and Bz-insensitive VR1 variant cation channels in fungiform TRCs demonstrate many functional similarities with VR1. Therefore, whether ethanol also modulates the apical Na⁺ flux through the VR1 variant non-specific cation channels in fungiform TRCs was investigated. The effect of ethanol (ETH) on the unilateral Na⁺ flux across the apical membrane of polarized fungiform TRCs was observed. Initially, a lingual epithelial preparation was perfused on the basolateral side with a Na⁺-free Ringer's solution and on the apical side with a Na⁺-free Ringer's solution containing 5 mM Bz. In the continuous presence of Bz, increasing apical Na⁺ concentration from 0 to 150 mM produced a reversible increase in F₄₉₀. An increase in F₄₉₀ indicates an increase in TRC [Na⁺]ᵢ. Perfusing the apical membrane with Ringer's solution containing 150 NaCl+5 mM Bz+10% ethanol (ETH) produced a bigger increase in F₄₉₀ relative to zero ethanol. Increasing ethanol concentration to 40% produced a further increase in F₄₉₀ relative to 10% ethanol. In contrast, perfusing the apical membrane with Na⁺-free Ringer's solution containing 10% or 40% ethanol induced no changes in F₄₉₀ relative to zero ethanol. These results indicate that ethanol at a concentration between 10% and 40% increases the unilateral Bz-insensitive Na⁺ flux across the apical membrane of fungiform TRCs in a dose-dependent manner.

Effect of ethanol on the unilateral apical Na⁺ flux in the presence of VR1 antagonists. To investigate if ethanol increases apical Na⁺ flux via the VR1 variant non-specific cation channel, further experiments were performed in the presence of VR1 antagonists, capsazepine (CZP) and SB-366791. In a lingual epithelial preparation, perfusing the apical membrane with Ringer's solution containing 150 mM NaCl+5 mM Bz+40% ETH+100 mM CZP produced a significantly smaller increase in F₄₉₀ relative to Ringer's solution containing 150 mM NaCl+5 mM Bz+40% ETH. The results further show that in the presence of 150 mM NaCl+5 mM Bz+40% ETH+100 mM CZP the magnitude of the increase in F₄₉₀ was same as that observed with 150 mM NaCl+5 mM Bz+10% ETH.

In another lingual epithelial preparation, perfusing the apical membrane with 150 mM NaCl+5 mM Bz+1 mM SB-366791 produced no increase in F₄₉₀ relative to zero Na⁺ concentration, and subsequently perfusing 150 mM NaCl+5 mM Bz+1 mM SB-366791+40% ETH produced no further increase in F₄₉₀ above baseline. These results indicate that SB-366791 not only completely inhibits the resting Bz-insensitive apical Na⁺ flux but also completely blocks the effects of ethanol on the Na⁺ flux. Similar results were seen in experiments on isolated TRCs.

In Vivo Studies

Effect of ethanol on CT responses to mineral salts. The results above in this Example 5 indicate that ethanol modulates the VR1 variant cation channel and increases apical Na⁺ flux. VR1 agonists and antagonists that modulated the apical membrane cation conductance and the apical Na⁺ flux in fungiform TRCs also modulated the CT responses to NaCl.

First, the CT responses to ethanol were monitored alone. The rat tongues were rinsed with distilled H₂O and then stimulated with ethanol at concentrations varying between 40% and 100% maintained at room temperature (23°). The CT response to ethanol was composed of only a transient phasic component. No sustained tonic component of the CT response was observed at any concentration of ethanol. The magnitude of the transient phasic response remained invariant with increasing ethanol concentration. Upon stimulating the tongue with ethanol solutions containing 0.1 mM SB-366791, a potent and specific blocker of VR1 variant channels, did not affect the transient phasic responses to ethanol relative to control.

That the apical VR1 variant cation channels in TRCs are activated by elevated temperature has been demonstrated. To test if ethanol responses are also modulated at elevated temperature, the tongue was stimulated with ethanol solutions maintained at 42° and the CT responses were monitored with reference to the H₂O rinse at 23°. Increasing the temperature to 42° had no effect of the CT response to 80% ethanol stimulation relative to 230. Transient phasic CT responses were also obtained with H₂O rinse following the stimulation with 100% ethanol. Taken together, the above results suggest that ethanol, by itself, produces only transient phasic CT responses. The magnitude of the transient phasic response was not affected by ethanol concentration, the presence of VR1 agonists (elevated temperature) or VR1 antagonists (SB-366791). In addition, the transient phasic CT responses are not specific to ethanol stimulation but are also observed after rinsing the tongue with distilled H₂O.

In the above experiment the effect of ethanol on the CT response was monitored in distilled H₂O. The next series of experiments investigated if CT responses to ethanol are altered when even a small amount of a mineral salt is present in a mixture with ethanol in the stimulating solution. In these experiments the tongue was initially rinsed with 10 mM KCl (R) and then stimulated with the stimulating solution containing 10 mM KCl+ethanol. The ethanol (ETH) concentration in the stimulating solution was varied between 20% and 60%. Data show that R+20% ETH did not increase the CT response above baseline relative to R alone. Stimulating with R+30% ETH produced only a transient phasic response. Stimulating with R+40% ETH produced a CT response composed of a transient phasic response that was followed by a sustained tonic response. Similarly, both phasic and tonic components of the CT response were observed following the stimulation of the tongue with R+50% ETH and with R+60% ETH. These results indicate that the CT response profile is altered when even a small amount of a mineral salt is present in a mixture with ethanol.

The constitutively active Bz-insensitive NaCl CT response has been shown to be insensitive to changes in external pH (pHₒ) and ATP. However, in the presence of a sub-threshold concentration of a VR1 agonist, the Bz-insensitive NaCl CT response became sensitive to pHₒ and ATP. The next series of experiments tested if a sub-threshold concentration of RTX modulates the effects of ethanol on the Bz-insensitive NaCl CT response. The effect of ethanol (ETH) stimulation at 23° and 42° in the presence and absence of 0.5 μM RTX was studied. Superfusing the tongue with 10 mM KCl+20% ethanol at 23° (R+20% ETH₂₃°) produced only a transient phasic response. Stimulating with 10 mM KCl+20% ETH at 42° (R+20% ETH₄₂°) gave a CT response composed of a phasic response followed by a sustained tonic phase. Stimulating with 10 mM KCl+20% ETH+0.5 μM RTX at 230 (R+20% ETH+RTX₂₃°) also produced a CT response comprising both a phasic component and a sustained tonic component. Increasing the temperature of the stimulating solution to 420 (R+20% ETH+RTX₄₂°) enhanced the magnitude of the tonic component by 50% relative to 23°. Stimulating the tongue with 10 mM KCl+40% ETH at 23° (R+40% ETH₂₃°) gave a CT response containing both a phasic and a tonic component. The magnitude of the response was enhanced when the stimulating solution was presented at 42° relative to 230. Stimulating with R+40% ETH+0.5 μM RTX enhanced the response at 23° and at 42° relative to its magnitude.

Stimulating the tongue with 10 mM KCl+0.5 μM RTX at 23° (R+RTX₂₃°) gave only a transient phasic response. This suggests that at low concentration of KCl, the RTX-induced increase in K⁺ flux across the membrane is not sufficient to enhance the magnitude of the CT response. However, in the presence of RTX, stimulating the tongue at 23° with either 40% ETH or 60% ETH increased the CT response in a dose dependent manner. Increasing the temperature of the stimulating solutions to 42° increased the magnitude of the CT response to 40% ETH and 60% ETH relative to 23°. Superfusing the tongue with stimulating solutions containing RTX+ETH+0.1 μM SB-366791 completely inhibited the tonic component of the CT response and only transient phasic responses were observed at 23° and 42°. Taken together, these results indicate that both RTX and elevated temperature modulate the effect of ethanol on the KCl CT response and these effects are inhibited in the presence of SB-366791.

To test if ethanol (ETH) affects the temperature threshold of the VR1 variant cation channel, CT responses were monitored while the temperature of the stimulating solution was varied between 23° and 55.5°. Stimulating the tongue with 10 mM KCl+60% ETH at 380 produced a sharp increase in the KCl CT response and gave maximum enhancement of the CT response at 41°. The KCl CT response decreased above 410. In contrast, stimulating with 10 mM KCl+60% ETH+0.5 μM RTX enhanced the CT response at 230 and at elevated temperatures without affecting the temperature threshold of the KCl CT response.

Next was investigated the effect of ethanol on the CT responses to 100 mM NaCl. The CT responses were monitored while the rat tongue was stimulated with a rinse solution (R) containing 10 mM KCl+ethanol (ETH; 20%-60%) and then with stimulating solutions containing 100 mM NaCl+10 mM KCl+5 mM Bz+ETH (20%-60%). In each case the ETH-induced change in the magnitude of the NaCl response was calculated as the difference between CT response with 100 mM NaCl+10 mM KCl+5 mM Bz+a particular concentration of ETH and the rinse response (10 mM KCl+the corresponding ETH concentration). Stimulating the tongue with 100 mM NaCl+5 mM Bz+ETH produced a dose-dependent increase in the magnitude of the Bz-insensitive NaCl CT response between 20% and 40% ethanol concentrations. The Bz-insensitive NaCl CT response achieved its maximum value between 40% and 50% ethanol concentration. The magnitude of the Bz-insensitive NaCl CT response decreased at 60% ethanol concentration. These results indicate that, resembling other VR1 agonists (RTX, CAP, and temperature), the relationship between the Bz-insensitive NaCl CT response and ethanol concentration is bell shaped. Thus ethanol, depending upon its concentration, acts both as an agonist and an antagonist of the Bz-insensitive salt taste receptor.

Studies with the VR1 KO Mice

To investigate whether ethanol modulates NaCl CT responses via the VR1 variant cation channel, the effect of ethanol was investigated on the CT responses in WT and VR1 KO mice. In WT mice, stimulating the tongue with 100 mM NaCl produced a CT response that is composed of a Bz-sensitive ENaC component and a Bz-insensitive component. Similar to the case in rats, in WT mice stimulation with 10 mM KCl+60% ETH (R+60% ETH) elicited a greater CT response relative 10 mM KCl. In addition, stimulating the tongue with 100 mM NaCl+5 μM Bz+60% ETH (N+Bz+60% ETH) inhibited the CT response relative to 100 mM NaCl+5 μM Bz(N+Bz).

In contrast, VR1 KO mice demonstrated no Bz-insensitive NaCl CT response component, no CT response to R+60% ETH, and demonstrated no effect of 60% ETH on the NaCl CT response or the Bz-insensitive NaCl CT response. These studies indicate that the taste response of the VR1 KO mice is insensitive to ethanol. The data from 3 WT and 3 VR1 KO mice show that in VR1 KO mice the magnitude of the CT response to R+60% ETH, N+Bz, N+Bz+60% ETH, and R+Bz+60% ETH was not significantly different from zero (p>0.05; N=3).

### EXAMPLE 6

Evodiamine, an indolequinazole alkaloid, is a non-pungent agonist of the VR1 receptor extracted from the fruits of Evodia rutaecarpa. It enhances the chorda tympani response to 100 mM NaCl+5 μM benzamil-beginning at 30 μM and reaching a maximum enhancement at 300 μM. At higher concentrations of evodiamine the responses decreases. At 3 mM the response is the same as the control response. At higher concentrations the response is inhibited. This is the same pattern observed with capsaicin and other pungent agonists.

100 μM evodiamine in the rinse solution (10 mM KCl) has no effect on the chorda tympani response, but 100 μM evodiamine+500 μM ATP gives further enhancement to the chorda tympani response of 100 mM NaCl+5 μM benzamil compared to the response of 100 mM NaCl+5 μM benzamil+100 μM evodiamine.

Evodiamine thus was identified as a non-pungent agonist of the amiloride-insensitive salt taste receptor.

### EXAMPLE 7

A Thiourea Containing Compound, 2-(3,4-dimethylbenzyl)-3-{[(4-hydroxy-3-methoxybenzyl)amino]carbothioyl}propyl pivalate. (Agonist 23).

Agonist 23 is a less-pungent agonist of the VR1 receptor compared with capsaicin. It enhances the CT response to 100 mM NaCl+5 μM benzamil at low concentration. At higher concentrations of agonist 23 the responses decrease. This is the same pattern observed with capsaicin and other pungent agonists. Agonist 23 is more effective at 41° than at 230 which is consistent with the properties of the more pungent agonists.

While the invention has been described in terms of its preferred embodiments, those skilled in the art will recognize that the invention can be practiced with modification within the spirit and scope of the appended claims.

1. A method of modulating salt taste in a mammal, comprising:
introducing to a mammalian taste receptor cell, a non-salty ligand.

2. The method of claim 1, wherein the ligand being introduced to a mammalian taste receptor cell is selected from the group consisting of: an agonist of a vanilloid receptor-1 (VR1) variant taste receptor; a non-vanilloid VR1 agonist; a non-vanilloid, non-VR1 agonist; cetylpyridinium chloride (CPC); dodecylpyridinium bromide; and a VR1 antagonist.

3. The method of claim 2, wherein the ligand is selected from the group consisting of: capsaicin; resiniferatoxin (RTX); piperine; 2-(3,4-dimethylbenzyl)-3-{[(4-hydroxy-3-methoxybenzyl)amino]carbothioyl}propyl pivalate (agonist 23).

4. The method of claim 2, wherein the agonist is a non-pungent agonist.

5. The method of claim 4, wherein the non-pungent agonist is selected from the group consisting of olvanil, capsiate and evodiamine.

6. The method of claim 2, wherein the ligand is a VR1 antagonist selected from capsazepine or SB366791; or the ligand is a VR1 agonist and is selected from the group consisting of: a compound including a vanilloid moiety; a compound with a vanilloid moiety replaced with a thiourea moiety; unsaturated dialdehydes; zingerones; indolequinazole alkaloids; and allyl isothiocyanates.

7. The method of claim 1, including isolating RNA necessary for producing VR1 and/or VR1-like channel and incorporating the isolated RNA in an assay system.

8. The method of claim 1, wherein the ligand is ethanol.

9. The method of claim 1, wherein salt taste is modified by a non-pungent VR1 ligand.

10. A method of modifying a salt transduction process in a mammal, comprising:
introducing a non-salty ligand into the salt transduction process, whereby the cation non-specific salt taste transduction process is modified.

11. The method of claim 10, wherein the non-salty ligand is introduced in a nanomolar or micromolar concentration.

12. The method of claim 10, wherein the salt transduction process is modified in a human.

13. A method of predicting whether a candidate ligand will enhance salt taste, comprising:
for a candidate ligand, determining pharmacology of the candidate ligand with respect to a capsaicin receptor, and
based on the pharmacology of the candidate ligand with respect to the capsaicin receptor, classifying the candidate ligand as likely or not likely to enhance a non-specific salt taste channel.

14. The method of claim 13, wherein the step of determining pharmacology of the candidate ligand with respect to a capsaicin receptor includes determining whether literature exists specifying the candidate ligand as an agonist or an antagonist for VR1 or TRPV1.

15. The method of claim 13, wherein location of a literature mention of the candidate ligand being an agonist for VR1 or TRPV1 signals a high likelihood that the candidate ligand will enhance a non-specific salt taste channel.

16. The method of claim 13, including finding no literature mention of the candidate ligand being an agonist or an antagonist for VR1 or TRPV 1, followed by a step of experimentally testing the candidate ligand.

17. The method of claim 16, wherein the experimental testing includes an in-vivo test on cultured cells and/or transfecting a molecular biological host with TRPV1 RNA.

18. The method of claim 17, wherein upon testing the candidate ligand on cultured mammalian dorsal root ganglion (DRG) cells, transient spiking is taken as an indicator of high probability that the candidate ligand will enhance a non-specific salt taste channel.

19. The method of claim 17, wherein upon transfecting a molecular biological host with TRPV1 RNA:
whether the candidate ligand elicits increase in cellular calcium is taken as an indicator of high probability that the candidate ligand will enhance a non-specific salt taste channel; and/or
whether the candidate ligand elicits increase in intracellular sodium is taken as an indicator of high probability that the candidate will enhance a non-specific salt taste channel; and/or
whether the candidate ligand elicits a change in membrane potential is taken as an indicator of high probability that the candidate ligand will enhance a non-specific salt taste channel.


SEQUENCE LISTING (2 sequences)

SEQ ID NO: 1 - DNA, 20 bp, Artificial
  synthetic oligonucleotide primer
tgaaaaacaccgttggggac

SEQ ID NO: 2 - DNA, 20 bp, Artificial
  synthetic oligonucleotide primer
gtagacgaacataaaccggc